(19) United States
(12) Patent Application Publication
(10) Pub. No.: US 2015/0307508 A1
(43) Pub. Date: Oct. 29, 2015

(54) Application of an alkaloid derived from a Chinese herbal for treatment of cancer by inhibiting cholesterol synthesis and fatty acid oxidation

(71) Applicant: Macau University of Science and Technology, Macau (CN)

(72) Inventors: Lai-Han LEUNG, Macau (CN); Liang LIU, Macau (CN); Xing-Xing FAN, Macau (CN); Jian-Lin WU, Macau (CN)

(21) Appl. No.: 14/645,406

(22) Filed: Mar. 11, 2015

Related U.S. Application Data
(60) Provisional application No. 61/983,465, filed on Apr. 24, 2014.

(51) Int. Cl. C07D 491/056

(52) U.S. Cl. CPC C07D 491/056

(57) ABSTRACT

The present invention discloses a method of treating Gefitinib-resistant non-small-cell lung cancer, comprising administering an effective amount of an alkaloid. A pharmaceutical composition comprising an alkaloid admixed with a pharmaceutical carrier for treating Gefitinib-resistant non-small-cell lung cancer is also disclosed therein.

### CROSS-REFERENCE TO RELATED APPLICATION

This application claims benefit under 35 U.S.C. §119(e) of U.S. Provisional Application having Ser. No. 61/983,465 filed 24 Apr. 2014, which is hereby incorporated by reference herein in its entirety.

### FIELD OF INVENTION

This invention relates to a novel treatment for treating Gefitinib-resistant non-small-cell lung cancer, in particular, a novel treatment involving the use of alkaloid.

### BACKGROUND OF INVENTION

Cholesterol is essential as building block of cell mass and is also essential for cell cycle to progress at G2 phase [1-3]. Cholesterol is also reported as an important biomolecule for the regulation of lipid raft function, arrangement of membrane receptors, sphingomyeline and ceramide, which may ultimately affect the membrane receptor tyrosine kinase activity [4]. For example, Epidermal Growth Factor Receptor (EGFR) is a commonly mutated gene in many types of cancer, including lung, brain and colon cancer etc, in which lung cancer is the leading cause of cancer deaths globally [12]. EGFR is aberrantly activated when the arrangement of the membrane lipid rafts are altered, which also directly affects tyrosine kinase inhibitor sensitivity, leading to drug resistance [4]. Recently, modulating cancer metabolism is emerging as an important strategy for new anti-cancer drug discovery [5]. Gefitinib, which is a tyrosine kinase inhibitor (TKI), can specifically inhibit EGFR as well as its downstream survival signaling pathway [13]. However, despite the initial significant responses to Gefitinib treatment, like other chemotherapeutic agents, patients acquire resistance to Gefitinib ultimately, and the median time to disease progression is just about 12 months [14]. The most common reason of Gefitinib resistance is the presence of additional EGFR mutation (EGFR^{L858R+T790M}), which accounts for over 49% of all the resistance cases.

Therefore, there is an urgent need to discover more effective agents as new candidate drugs for Gefitinib -resistant NSCLC patients.

### SUMMARY OF INVENTION

In the light of the foregoing background, it is an object of the present invention to provide an alternate treatment for Gefitinib-resistant non-small-cell lung cancer.

Accordingly, the present invention, in one aspect, relates to a method of treating Gefitinib-resistant non-small-cell lung cancer, comprising administering an effective amount of an isoquinoline alkaloid.

In one embodiment, the isoquinoline alkaloid is berberine. In a further embodiment, the Gefitinib-resistant non-small-cell lung cancer is induced by EGFR^{L858R+T790M} mutation.

In another aspect of this invention, the present invention provides a pharmaceutical composition comprising an isoquinoline alkaloid admixed with a pharmaceutical carrier for treating Gefitinib-resistant non-small-cell lung cancer.

In one embodiment, the isoquinoline alkaloid is berberine. In a further embodiment, the Gefitinib-resistant non-small-cell lung cancer is induced by EGFR^{L858R+T790M} mutation.

In another aspect of this invention, a dietary supplement comprising an isoquinoline alkaloid admixed with a pharmaceutical carrier for treating Gefitinib-resistant non-small-cell lung cancer is provided.

In one embodiment, the isoquinoline alkaloid is berberine.

In a further aspect of this invention, the present invention provides a pharmaceutical composition comprising an isoquinoline alkaloid admixed with a pharmaceutical carrier for regulating lipid metabolism or cholesterol synthesis.

In one embodiment, the isoquinoline alkaloid is berberine.

### BRIEF DESCRIPTION OF FIGURES

FIGS. 1A-1E show the anticancer effect exerted by berberine through arresting cell cycle in Gefinitib resistant cell lines in H1975 cells and in H1650 cells respectively.

FIGS. 2A-2D show the results of a study on the anticancer mechanism of berberine in Gefinitib resistant cell lines.

FIG. 3 shows the thin-layer chromatography spectrum of H1975 and H1650 cells treated by berberine.

### DETAILED DESCRIPTION OF THE PREFERRED EMBODIMENTS

Berberine (BBR) is known for having an effect of lowering cholesterol and anti-diabetics. The anti-cancer effect of BBR has also been reported; however, the treatment mechanism thereof is unclear. BBR has been used as traditional medicine and dietary supplement for a long time in Chinese history, and has shown some activities against fungal, yeast, parasites, and bacterial infections [6, 7]. However, there are few researches focusing on the anticancer effect of berberine on NSCLC by modulating cholesterol synthesis and fatty acid oxidation, especially on Gefitinib resistant NSCLC.

A panel of lung cancer cell lines was used as tools to intensively study the new role of berberine on Gefitinib resistant NSCLC. The anti-cancer activity of berberine on six different NSCLC cell lines and the anti-cancer mechanism thereof were studied. This is the first time to show that berberine has specific cytotoxicity effect on Gefitinib resistant cell lines. It is most effective in H1975 and H1650 cell lines which originally harbor EGFR activating mutation but are resistant to Gefitinib. It was also novel to find that the anti-cancer mechanism of berberine is related to modulation of lipid metabolism, resulting in cell cycle arrest. Overall, findings in this invention suggest a new application of berberine for treating Gefitinib NSCLC and new anti-cancer mechanism by modulating lipid metabolism.

### Example 1

### Study of Berberine Against Six NSCLC Cell Lines

This example described the BBR-induced cytotoxicity effect in six NSCLC cell lines utilizing MTT cytotoxicity assay. A549 is wild type EGFR and BEAS-2B is normal lung epithelial cells. H1975 and H1650 are Gefitinib-resistant NSCLC cell lines with mutations as EGFR^{L858R+T790M} and EGFR^{Exon19 deletion}. H1819 has EGFR amplification and HCC827 has EGFR^{exon 19 deletion}, and both of which are Gefitinib sensitive. The IC₅₀ values of each cell line after BBR treatment for 72 hrs are shown in Table 1.

TABLE 1

| | IC₅₀ value after 72 hrs of | |
|---|---|---|
| Cell lines | treatment (μM) | Gefitinib Sensitivity |
| H1975 | 2.92 ± 0.49 | Resistant |
| H1650 | 6.00 ± 2.78 | Resistant |
| H1819 | 6.51 ± 2.09 | Moderately sensitive |
| HCC827 | 8.90 ± 4.40 | Sensitive |
| A549 (EGFR^{Wild type}) | 11.20 ± 3.98 | Moderately sensitive |
| BEAS-2B (Normal lung | 24.67 ± 12.39 | Resistant |
| epithelial cell) | | |

Conclusion: From the results shown in Table 1, BBR has cytotoxicity effect in human NSCLC cell lines, especially in H1975 (EGFR^{L858R+T790M}) and H1650 (EGFR^{Exon19 deletion}) cell lines; while it shows relatively low cytotoxicity effect in BEAS-2B (normal lung epithelial cells) and A549 (EGFR^{Wild type}) cell lines. Thus, BBR is shown to be effective in treating Gefitinib-resistant non-small-cell lung cancer.

### Example 2

### Study of the Anticancer Effect of Berberine in H1975 and H1650 Lung Cancer Cells Through Arresting Cell Cycle

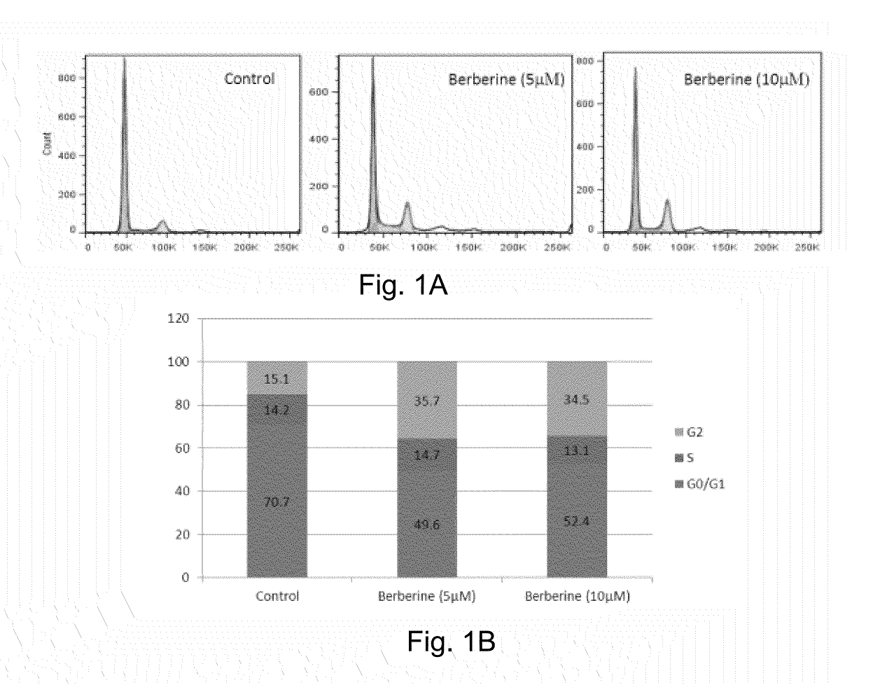

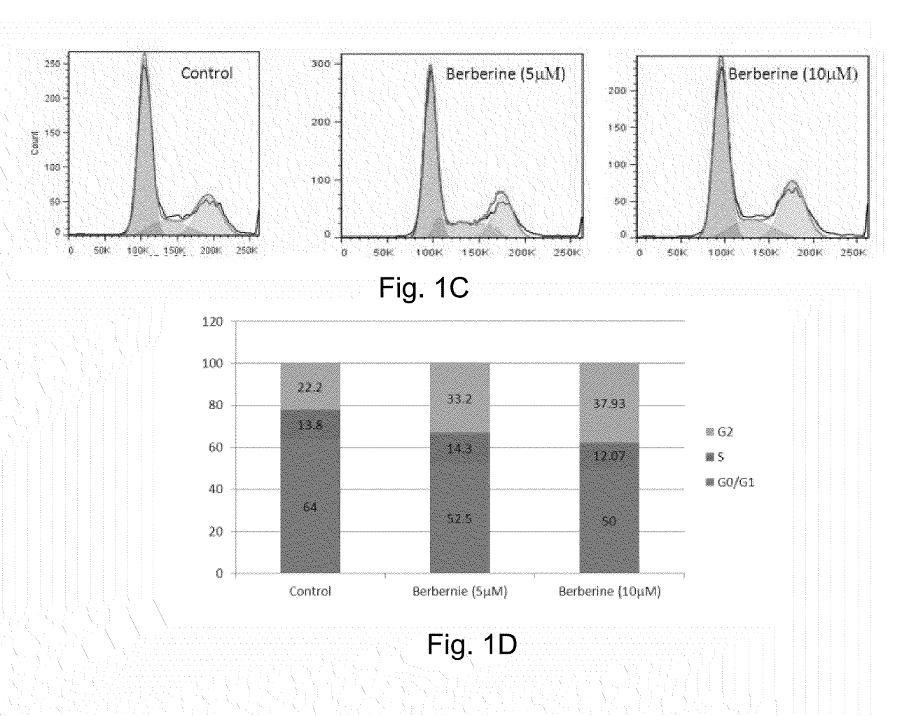

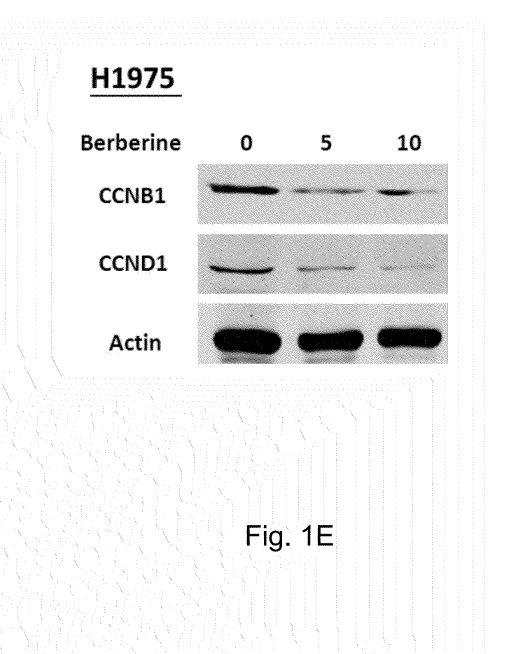

The anticancer effect of berberine against two most BBR sensitive cell lines are further studied in Example 8, namely H1975 (EGFR^{L858R+T790M}) and H1650 (EGFR^{Exon19 deletion}). Upon treatment with berberine at a concentration of 5 μM/10 μM for 72 hrs, the G2 cell cycle of H1975 and H1650 cells was observed and shown in FIGS. 1 A to 1 D. FIG. 1A shows the cell cycle analysis of H1650 in the groups of control, 5 μM and 10 μM berberine treatment, while FIG. 1B shows that, on comparing with the control group, the percentages of G0/G1 phase in H1650 were decreased by 21.1% and 18.3% respectively after the treatment with berberine at concentrations of 5 μM and 10 μM. Correspondingly, G2 phase of the groups of 5 μM and 10 μM berberine treatment significantly were increased by 20.6% and 19.4% respectively as compared with the control group (n=3).

FIG. 1C shows the cell cycle analysis of H1975 in the groups of control, 5 μM and 10 μM berberine treatment, and FIG. 1D shows that, on comparing with the control group, the percentages of G0/G1 phase in H1975 were decreased by 11.5% and 14% respectively after the treatment with berberine at concentrations of 5 μM and 10 μM. Correspondingly, G2 phase of the groups of 5 μM and 10 μM berberine treatment significantly were increased by 11% and 15.73% respectively as compared with the control group (n=3).

FIG. 1E shows the Western blot analysis of the protein levels of CCNB1 and CCND1 in H1975 cells, indicating that cell cycle promoting genes CCNB1 and CCND1 were down-regulated after 72 hrs of BBR treatment in lung cancer cells. This result further suggests that cell cycle arrest is due to down-regulation of CCNB1 and CCND1 by BBR.

Conclusion: On comparing with the untreated group, the percentages of cells in G2 of H1975 cells have increased 11% and 15.7% in berberine 5 μM and 10 μM groups respectively. The percentages of cells in G2 of H1650 cells have also increased for more than 10% in both berberine 5 μM and 10 μM groups as compared with the control group.

### Example 3

### Study of Anticancer Mechanism of Berberine in H1975 And H1650 Cancer Cell Lines

In Example 3 Berberine anticancer mechanism in H1975 and H1650 cancer cell lines was studied to see if cholesterol synthesis or fatty acid oxidation pathways is involved.

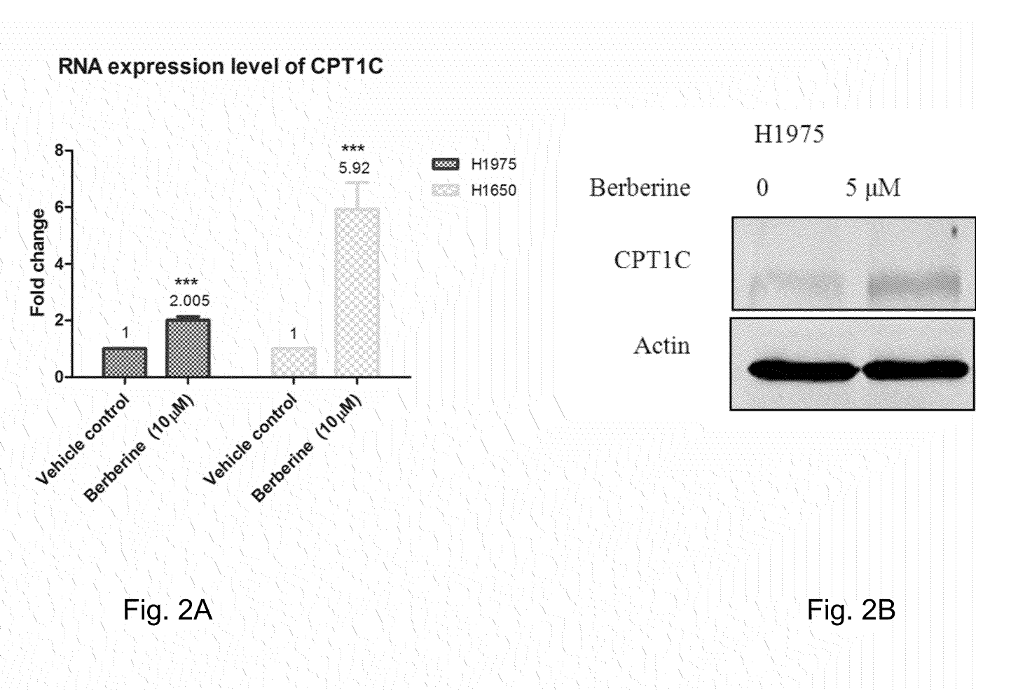

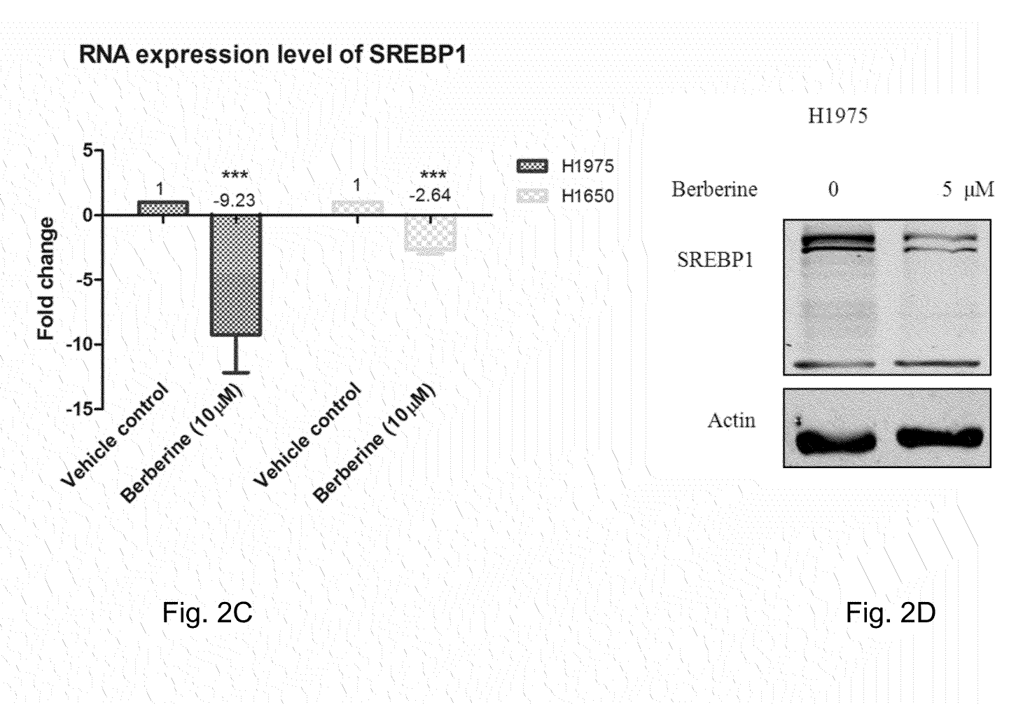

The RNA expression levels of CPT1C and SREBP1 in both H1975 and H1650 cell lines after being treated with 10 μM BBR for 24 hrs were analyzed by Real-time PCR analysis and Western blot analysis, as shown in FIGS. 2A-2D. FIG. 2A shows that after 40 cycles, the RNA expression level of CPT1C in H1975 and H1650 cells were increased by 2.005 and 5.92 times respectively as compared with the vehicle control. The result of study of H1975 cells was further supported by the Western blot shown in FIG. 2B.

FIG. 2C shows that after 40 cycles, the RNA expression level of SREBP1 in H1975 and H1650 cells were decreased for 9.23 and 2.64 times respectively as compared with the vehicle control. The result of study of H1975 cells was further supported by the Western blot analysis shown in FIG. 2D.

Conclusion: The results of Example 3 shows that berberine can up-regulate the fatty acid oxidation gene: CPT1C, and at the same time down-regulate the fatty acid synthesis gene SREBP1. These two genes are key enzymes involved in lipid metabolism and cholesterol synthesis.

### Example 4

### Study of Thin-Layer Chromatography Spectrum of the Overall Lipid Levels in H1975 and H1650 Cells Treated by BBR

Cells were scraped from culture dish and resuspended in 2 ml PBS containing 1× protease inhibitor and 1 mM PMSF. Total cellular lipid was extracted by adding 4 ml chloroform/methanol (2:1). The mixture was vortexed for 1 min and centrifuged at 1500 g×5 min. The organic phase was collected and another 2.5 ml choloroform was added to the residual aqueous phase; the mixture was vortexted and centrifuged at 1500 g×5 min again. The organic phase was collected together with the previous extraction. Thin layer chromatography (TLC) was performed by spotting of the cellular total lipid extract to a 5×10 cm silica gel aluminum sheet and developed with hexane/diethyl ether/acetic acid (80:20:2). Lipids were visualized with iodine vapor and imaged using a desktop scanner

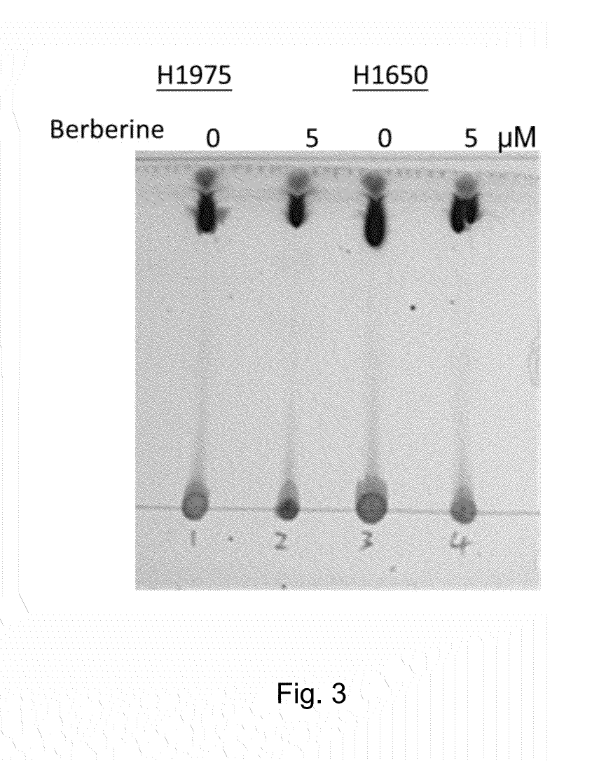

The overall lipid levels in H1975 and H1650 treated by Berberine were studied in this example. H1975 and H1650 cells were treated with 5 μM BBR for 72 hours and then total cellular lipid was extracted. The thin-layer chromatography spectrum was shown in FIG. 3.

Conclusion: The result indicates that BBR can lower the overall lipid levels in both H1975 and H1650 lung cancer cells.

### SUMMARY

This is the first study to show that berberine exerted cytotoxic effect and cell cycle arrest effects on cancer cells by inhibiting cholesterol synthesis and fatty acid oxidation. Taken together, these results indicate that berberine could be used as a candidate agent against Gefitinib-resistant NSCLC patients, especially for the group of patients with EGFR^{L858R+T790M} mutation which represents 49% of all Gefitinib resistance cases.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### REFERENCES

1. Fernandez, C., et al., Cholesterol is essential for mitosis progression and its deficiency induces polyploid cell formation. Experimental cell research, 2004. 300(1): p. 109-20.
2. Martinez-Botas, J., et al., Dose-dependent effects of lovastatin on cell cycle progression. Distinct requirement of cholesterol and non-sterol mevalonate derivatives. Biochimica et biophysica acta, 2001. 1532(3): p. 185-94.
3. Suarez, Y., et al., Sterol stringency of proliferation and cell cycle progression in human cells. Biochimica et biophysica acta, 2005. 1734(2): p. 203-13.
4. Filosto, S., et al., EGF receptor exposed to oxidative stress acquires abnormal phosphorylation and aberrant activated conformation that impairs canonical dimerization. PloS one, 2011. 6(8): p. e23240.
5. Hanahan, D. and R. A. Weinberg, Hallmarks of cancer: the next generation. Cell, 2011. 144(5): p. 646-74.
6. Berberine. Alternative medicine review : a journal of clinical therapeutic, 2000. 5(2): p. 175-7.
7. Kulkarni, S. K. and A. Dhir, Berberine: a plant alkaloid with therapeutic potential for central nervous system disorders. Phytotherapy research : PTR, 2010. 24(3): p. 317-24.
8. Li, J., et al., Berberine suppresses androgen receptor signaling in prostate cancer. Molecular cancer therapeutics, 2011. 10(8): p. 1346-56.
9. Yu, F. S., et al., Berberine inhibits WEHI-3 leukemia cells in vivo. In vivo, 2007. 21(2): p. 407-12.
10. Mantena, S. K., S. D. Sharma, and S. K. Katiyar, Berberine inhibits growth, induces G1 arrest and apoptosis in human epidermoid carcinoma A431 cells by regulating Cdki-Cdk-cyclin cascade, disruption of mitochondrial membrane potential and cleavage of caspase 3 and PARP. Carcinogenesis, 2006. 27(10): p. 2018-27.
11. Eom, K. S., et al., Berberine induces G1 arrest and apoptosis in human glioblastoma T98G cells through mitochondrial/caspases pathway. Biological & pharmaceutical bulletin, 2008. 31(4): p. 558-62.
12. Gansler, T., et al., Sixty years of CA: a cancer journal for clinicians. CA: a cancer journal for clinicians, 2010. 60(6): p. 345-50.
13. Ono, M. and M. Kuwano, Molecular mechanisms of epidermal growth factor receptor (EGFR) activation and response to gefitinib and other EGFR-targeting drugs. Clinical cancer research: an official journal of the American Association for Cancer Research, 2006. 12(24): p. 7242-51.
14. Sequist, L. V., et al., Genotypic and histological evolution of lung cancers acquiring resistance to EGFR inhibitors. Science translational medicine, 2011. 3(75): p. 75ra26.

What is claimed is:

1. A method of treating Gefitinib-resistant non-small-cell lung cancer, comprising administering an effective amount of an isoquinoline alkaloid.

2. The method of claim 1, wherein said isoquinoline alkaloid is berberine.

3. The method of claim 1 wherein said Gefitinib-resistant non-small-cell lung cancer is induced by EGFR^{L858R+T790M} mutation.

4. A pharmaceutical composition comprising an isoquinoline alkaloid admixed with a pharmaceutical carrier for treating Gefitinib-resistant non-small-cell lung cancer.

5. The pharmaceutical composition of claim 4, wherein said isoquinoline alkaloid is berberine.

6. The pharmaceutical composition of claim 4 wherein said Gefitinib-resistant non-small-cell lung cancer is induced by EGFR^{L858R+T790M} mutation.

7. A dietary supplement comprising an isoquinoline alkaloid admixed with a pharmaceutical carrier for treating Gefitinib-resistant non-small-cell lung cancer.

8. The dietary supplement of claim 7, wherein said isoquinoline alkaloid is berberine.

9. A pharmaceutical composition comprising an isoquinoline alkaloid admixed with a pharmaceutical carrier for regulating lipid metabolism or cholesterol synthesis.

10. The pharmaceutical composition of claim 9, wherein said isoquinoline alkaloid is berberine.
